(19) United States
(12) Patent Application Publication
(10) Pub. No.: US 2024/0185056 A1
(43) Pub. Date: Jun. 6, 2024

(54) AN APPARATUS FOR ENHANCING LONGEVITY AND METHOD FOR ITS USE

(71) Applicant: Oceandrive Ventures, LLC, Rio Grande, PR (US)

(72) Inventor: Jeffrey Gladden MD FACC, Rio Grande, PR (US)

(73) Assignee: Oceandrive Ventures, LLC, Rio Grande, PR (US)

(21) Appl. No.: 18/073,064

(22) Filed: Dec. 1, 2022

(51) Int. Cl. G06N 3/08; G16H 50/20

(52) U.S. Cl. CPC G06N 3/08; G16H 50/20

(57) ABSTRACT

An apparatus for enhancing longevity, wherein the apparatus includes at least a processor and a memory communicatively connected to the processor, the memory containing instructions configuring the at least a processor to receive a longevity measurement pertaining to a user and identify a compositional longevity parameter as a function of the longevity measurement. The memory containing instructions further configuring the processor to generate a symphonic longevity plan pertaining to the user as a function of the compositional longevity parameters, wherein generating further includes training a machine-learning process using a compositional longevity training data, wherein the compositional longevity training data contains a plurality of inputs containing compositional longevity parameters correlated to a plurality of outputs containing symphonic longevity plan.

### FIELD OF THE INVENTION

The present invention generally relates to the field of health technologies. In particular, the present invention is directed to an apparatus for enhancing longevity and method for its use.

### BACKGROUND

There may be no upper limit to human lifespan. An advanced, self-use, and portable system/method/apparatus with a flexible and highly personalized plan is necessary for people in preventing disease, improving overall health, and enhancing longevity. Existing solutions are not satisfactory.

### SUMMARY OF THE DISCLOSURE

In an aspect, an apparatus for enhancing longevity, wherein the apparatus includes at least a processor and a memory communicatively connected to the processor, the memory containing instructions configuring the at least a processor to receive a longevity measurement pertaining to a user and identify a compositional longevity parameter as a function of the longevity measurement. The memory containing instructions further configuring the processor to generate a symphonic longevity plan pertaining to the user as a function of the compositional longevity parameters, wherein generating further includes training a machine-learning process using a compositional longevity training data, wherein the compositional longevity training data contains a plurality of inputs containing compositional longevity parameters correlated to a plurality of outputs containing symphonic longevity plan.

In another aspect, a method for enhancing longevity is shown, the method includes receiving, using a processor, a longevity measurement pertaining to a user. The processor identifies compositional longevity parameter as a function of the longevity measurement. Additionally, the processor generates a symphonic longevity plan as a function of the compositional longevity parameter, wherein the generation further includes training a machine-learning process using a compositional longevity training data, wherein the compositional longevity training data contains a plurality of inputs containing compositional longevity parameters correlated to a plurality of outputs containing symphonic longevity plan.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of an apparatus for enhancing longevity;

FIG. 2 is a block diagram of an exemplary machine-learning process;

FIG. 3 is a block diagram of an exemplary embodiment of a compositional longevity database;

FIG. 4 is a diagram of an exemplary embodiment of neural network;

FIG. 5 is a diagram of an exemplary embodiment of a node of a neural network;

FIG. 6 is a flow diagram of an exemplary method of enhancing longevity; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus and method for enhancing longevity. The apparatus may comprise at least a processor and a memory communicatively connected to the processor. The processor then may be configured to receive a longevity measurement pertaining to a user. The processor then may identify a compositional longevity parameter as a function of the longevity measurement. A symphonic longevity plan is then generated by the processor for the user. Generation of the symphonic longevity plan includes training a machine-learning process using a compositional training data, wherein the compositional longevity training data contains a plurality of inputs containing compositional longevity parameters correlated to a plurality of outputs containing symphonic longevity plan.

Aspects of the present disclosure can also be used to generate symphonic longevity plan in a bottom-up approach. In some embodiments, symphonic longevity plan may be configured to eliminate disease symptoms of the user. In some embodiments, symphonic longevity plan may be configured to reduce a recurrence rate of such symptoms. Exemplary embodiments illustrating aspects of the present disclosure are described below in the context of several specific examples.

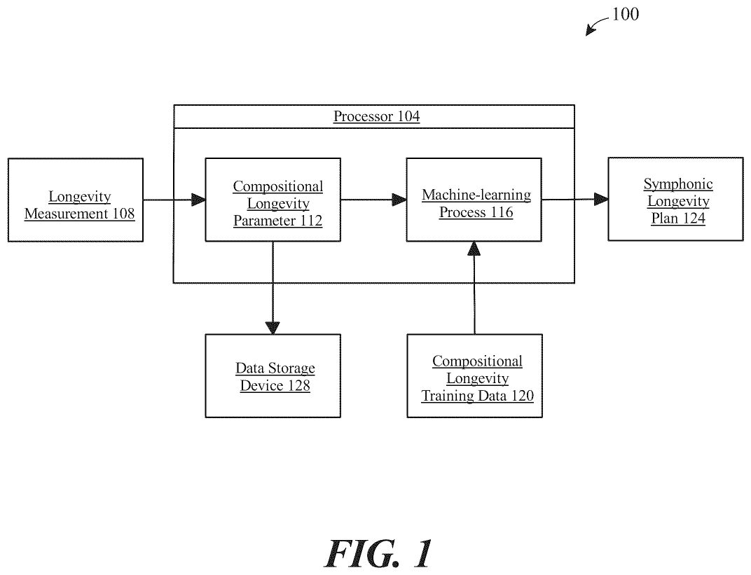

Referring now to FIG. 1, an exemplary embodiment of an apparatus 100 for enhancing longevity is illustrated. Apparatus 100 includes a processor 104. Processor 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented using a “shared nothing” architecture in which data is cached at the worker, in an embodiment, this may enable scalability of apparatus 100 and/or computing device.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

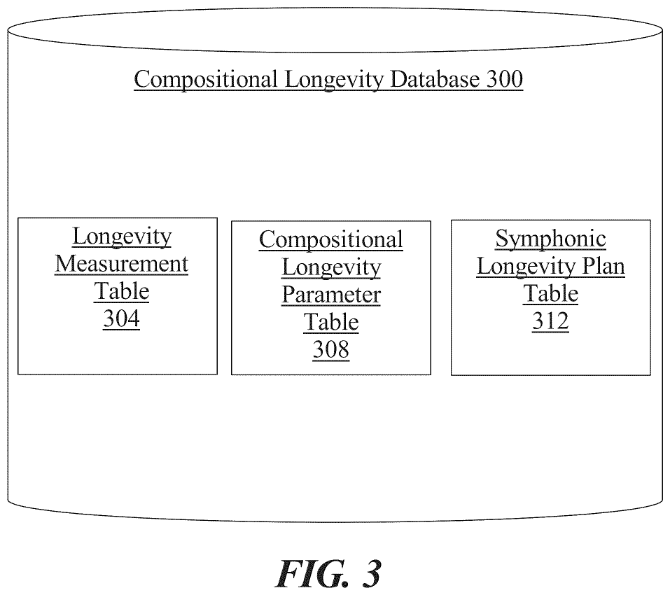

Continuing in reference to FIG. 1, processor 104 may receive, pertaining to a user, a longevity measurement 108. As used in this disclosure, “receive” from a user means accepting, collecting, or otherwise receiving input from a user and/or device. A “longevity measurement”, as used in this disclosure, is data that relates to the user's system or performance metrics. As used in the current disclosure, “systems” are biological systems within a human body. A user's systems may include, but is not limited to, the circulatory, nervous, skeletal, respiratory, reproductive, endocrine, integumentary, renal, digestive, and muscular systems. Each organ may have one or more specialized role in the body and is made up of distinct tissues. Additionally, a user's systems may include the heart, lungs, kidneys, or any other organ system. This may also include the components of a given system. In a non-limiting example, the lungs may be component of the respiratory system. “Performance metrics,” as used in this disclosure, are numeric or linguistic measurements of a user's ability to perform one or more predetermined tasks. Predetermined tasks may include, but are not limited to, walking, running, squatting, jumping, lifting, sleeping, eating, thinking, learning, and the like. In an embodiment, the longevity measurement 108 may include information collected from a standard health screening. A standard health screening may include, but is not limited to, tests like blood test, hearing test, vision test, height, weight, and body mass index (BMI), and the like. In other embodiment, the longevity measurement 108 may include medical history, diet, exercise, sleep, time, and geographical location data of the user. In an embodiment, longevity measurement 108 may be stored in a data storage device 128, such as a compositional longevity database 300 disclosed with reference to FIG. 3.

With continued reference to FIG. 1, longevity measurement 108 may include one or more measurable longevity markers. As used in this disclosure, a “longevity marker” is a biomarker that represent one or more aspects of the longevity of the user. As used in the current disclosure, a “biomarker” is a biological molecule found in blood, other body fluids, or tissues that is a sign of a normal or abnormal process, or of a condition or disease. A biomarker may be used to see how well the body responds to a treatment for a disease or condition. A measurable biomarker may include, but is not limited to, yH2A. X immunohistochemistry, Leukocyte telomere length, MIR31HG, p16INK4a, Senescence-associated secretory phenotype (SASP) proteins, Measures of DNA methylation, SIRT1, SIRT2, SIRT3, SIRT6, SIRT7, Dosage of circulating microRNAs (miR-34a, MiR-21, miR-126-3p, miR-151a-3p, miR-181a-5p, miR-1248), P31 MRI spectroscopy, growth differentiating factor 15 (GDF15), Target of rapamycin (TOR), Protein carbonylation, Advanced glycation end products, Insulin-like growth factor (IGF-1), HGBA1c, IL-6, TNF-α, CRP (C-reactive protein), and TNFRII (tumor necrosis factor-α RII). These biomarkers may be measured using various pathways including, but is not limited to, DNA repair mechanisms, DNA modifications, telomere length, markers of DNA damage response, telomerase activity, senescent markers in blood and tissue, DNA methylation, histone acetylation, noncoding RNA, autophagy markers, chaperon proteins, proliferative capacity in vitro, growth hormone axis, and metabolism alterations. In an embodiment, the longevity markers may include, but it is not limited to, telomerase length/age, mitochondrial function, DNA repair, function of STEM cells, epigenetic rate of aging, senescent cell burden, immune system function, inflammation, mTOR AMPK balance, epigenetic urine, senescent cell SASP, and the like.

With continued reference to FIG. 1, processor 104 may be configured to identify a compositional longevity parameter 112 as a function of the longevity measurement 108. As used in this disclosure, a “compositional longevity parameter” is an element of data that indicates age of the systems of the user in different categories. In some cases, categories of a user's systems may include, without limitation, life energy, health, longevity, performance, and the like thereof. In an embodiment, compositional longevity parameter 112 may be stored in a data storage device 128, such as compositional longevity database 300 disclosed with reference to FIG. 3. Processor 104 may generate the compositional longevity parameter 112 using one or more machine-learning processes trained with longevity training data. In some cases, machine-learning processes may be machine-learning process 116 described below in this disclosure. Longevity training data is a plurality of data entries containing a plurality of inputs that are correlated to a plurality of outputs for training a processor by a machine-learning process to align, classify, and determine the compositional longevity parameters to/of the user. In an embodiment, the inputs of the longevity training data may contain one or more longevity measurements and the outputs of the longevity training data may contains one or more compositional longevity parameters. In some cases, longevity training data may be obtained from a data storage device 128. In a non-limiting example, data storage device 128 may be a compositional longevity database 300. Compositional longevity database 300 is described in further detail with reference to FIG. 3. In some embodiments, longevity training data may include manually labeled data. As a non-limiting example, longevity measurements 108 and/or compositional longevity parameters 112 may be manually collected and labeled by the user and/or a medical professional. In some embodiments, machine-learning process 116 may be used to classify longevity measurements 108 to compositional longevity parameters 112; this, as non-limiting examples, may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

With continued reference to FIG. 1, in some embodiments, the compositional longevity parameter 112 may include a life energy measurement. A “life energy measurement”, as used in this disclosure, is a numeric measurement used to represent, measure, calculate, monitor, experiment, and/or evaluate the amount or level of the life energy flowing inside the energy meridian of a human or life body. As used in the current disclosure, “Life energy” is a form of energy related to but is not limited to, the brain, lungs, liver, bladder, kidneys, heart, stomach, and intestines in the human body. The Life energy measurement may also include an evaluation of the mental health status of human or life. This may include an evaluation of the mental health of human or life regarding overall energy, safety, wisdom, spiritual health, overall joy, relationship health, growth mindset, and overall mental health. In some cases, life energy measurement may be measured and/or derived by evaluation of telomeres, mitochondria, proteomic measures and/or evaluations, glycans measures and/or evaluations, oncogenic measures and/or evaluations, determinations of epigenetic rates of aging, epigenetic urine measures and/or evaluations, DNA repair measures and/or evaluations, epigenetic intrinsic measures and/or evaluations, epigenetic extrinsic measures and/or evaluations, mTor AMPK balance measures and/or evaluations, NAD measures and/or evaluations, NADH measures and/or evaluations, determinations regarding prevalence, health or other qualities of stem cells, senescent cell burden measures and/or evaluations, senescent cell SASP measures and/or evaluations, immune system measures and/or evaluations, and/or inflammation measures and/or evaluations. In an embodiment, the calculation of the life energy measurement may encompass an evaluation from a mental health professional and user inputs. In another embodiment, the calculation of the life energy measurement may include a life energy comparison metric. A “life energy comparison metric,” as used in this disclosure, is a comparison of the user's current state of the life energy compared to the average or/and healthy state of the life energy of one or more similar situated (age, gender, weight, height, and the like) users.

With continued reference to FIG. 1, in some embodiments, the compositional longevity parameter 112 may include a health age related to the user. As used in this disclosure, a “health age” is a comprehensive measurement related to the overall health status of the user. The health age may be generated as a function of the life energy measurement of the user. In a non-limiting example, a 50-year-old user with a high life energy measurement, wherein the high life energy measurement may be equivalent to a 25 years old. 50-year-old user may have a compositional longevity parameter 112, wherein the compositional longevity parameter 112 may include a health age of 25. In an embodiment, the health age may include, but is not limited to, the evaluation of the gender, weight, substance abuse, family health history, life energy, overall health, performance metrics and the like. In other embodiment, the health age may be measured using one or more patient specific metrics. A “patient specific metric” is specific performance metric based on the current physical or mental health condition of the user. The patient specific metric may include, but is not limited to, current sleep status, vascular and anatomic function, lung health, sex hormone balance, sexual function, brain function, brain anatomy, bone health, muscle function, joint function, ligament function, and tendon function. In other embodiment, the health age may also include evaluation of one or more lifestyle choices of a user. As used in this disclosure, a “lifestyle choice” is a personal and conscious decision to perform a behavior that may increase or decrease the risk of injury or disease, or/and the overall quality of life of a human being. In a non-limiting example, a healthy lifestyle choice can benefit both physically and mentally in everyday life of the user. The lifestyle choice may include, but is not limited to, exercising regularly, maintaining a healthy body weight, avoiding stationary sitting, avoiding sugar, eating more vegetables and fruits, drinking more water, going to bed early, quitting smoking and the like. In an embodiment, calculation of the health age may include a health age comparison metric. A “health age comparison metric,” as used in this disclosure, is a comparison of the user's current overall health status compared to the average or/and healthy overall health status of one or more similarly situated (age, gender, weight, height, and the like) users.

With continued reference to FIG. 1, in some embodiments, the compositional longevity parameter 112 may include a longevity age related to the user. As used in this disclosure, a “longevity age” is a biological measurement related to the user representing the length or the duration of maintaining user's health age. In an embodiment, a longevity age may be the time from the users current age until system failure and/or death. The longevity age may be calculated as a function of the health age and the life energy measurement of the user. The longevity age may be determined by a rate of aging of the user. As used in this disclosure, a “rate of aging” is an indication of the rate of change of age as a function of the current status of a given system. Rate of aging may be used to determine how quickly a given system is aging. Rate of aging may also apply to a given component of a system. Rate of aging may be reflected in terms of a ratio or a fraction. In a non-limiting example, a user with a high rate of aging may have a lower longevity age, because the user may be aging quickly. In another non-limiting example, a user with a low rate of aging may have a higher longevity age, because the user may be aging slowly. The difference between a first and a second compositional longevity parameter as numerator and the time between evaluations as denominator. Wherein the first compositional longevity parameter (CLP1) is taken at the first evaluation and the second compositional longevity parameter (CLP2) is taken at the second evaluation.

$$\\ Rate of Aging = \frac{C L {P}_{1} - C L {P}_{2}}{Time between evaluations}$$

For example, a 25-year-old user's overall health status is similar to a 35-year-old on the first evaluation, therefore the user may have a compositional longevity parameter of 10. During the second evaluation, approximately 1 year later, the 25-year-old user may have an overall health status similar to 40 year-old, therefore, the user may have a compositional longevity parameter of 15. The rate of aging of the 25-year-old user will be approximately −5. This may mean that the user's systems are aging at 5× the rate of a normal person. In reference to the rate of aging, a value ranging from −1 to 1 may be considered healthy. In an embodiment, the further the rate of aging is away from 0, the faster the user is aging either positively or negatively. Positive ageing is when the system is getting progressively healthier and thus more comparable to a younger person. Negative aging is when the system is getting progressively older and thus more comparable to an older person.

With continue reference on FIG. 1, the compositional longevity may include a performance age related to the user. As used in this disclosure, a “performance age” is a biological measurement that measures the user's ability to perform one or more predetermined tasks. Abilities that may be tested in evaluating a performance age of a user may include but not limited to the user's speed, flow state, agility, resilience, strength, reflection, both static and dynamic balance, recovery, flexibility, endurance, and the like. In an embodiment, the performance age may be generated as a function of the longevity age. In a non-limiting example, a user with a high longevity age may have a high performance age, since the user may have a longer duration of maintaining a health age. In another non-limiting example, a user with a low longevity age may have a low performance age, since the user may have a shorter duration of maintain a health age. In another embodiment, performance age may include a performance age comparison metric. A “performance age comparison metric,” as used in this disclosure, is a comparison of the user's current ability to perform one or more tasks compared to the average or/and expected ability to perform the same tasks of one or more similar situated (age, gender, weight, height, and the like) users.

With continue reference to FIG. 1, the processor 104 may be configured to generate a symphonic longevity plan 124 as a function of the compositional longevity parameter 112. A “symphonic longevity plan,” as used in this disclosure, is a list of detailed descriptions or instructions that help to enhance the longevity, or otherwise reverse aging of the user. Reversing aging may include reversing age such as, without limitation, health age, longevity age, performance age, and the like thereof. In an embodiment, the symphonic longevity plan 124 may include a set of corrective measures configured to strategically balance or/and improve the compositional longevity parameter 112 of the user. The symphonic longevity plan 124 may identify one or more problematic areas of the user based on user's compositional longevity parameters 112. As used in this disclosure, a “problematic area” is a field, aspect, or system associated with one or more values or measurements that is below average in the user. In an embodiment, the problematic area of the user may be an aspect where user lack essential elements or/and need to be improved. In another embodiment, the problematic area may be an unhealthy system of the user. For example, an area x of the user that is currently evaluated through the compositional longevity parameter (CLP_{current}), with measurements that are discovered to be less than the average (avg) measurements within the same area of one or more healthy users that evaluated through the compositional longevity parameter (CLP_{healthy}), is determined as a problematic area.

∀x∈CLP_{current}:x<avg(CLP_{healthy})

In some embodiments, compositional longevity parameter 112 may include an assigned weight. As used in this disclosure, an “assigned weight” is a statistical weight assigned to compositional longevity parameter 112 based on a focus. In a non-limiting example, compositional longevity parameter with larger assigned weight may be more important than a compositional longevity parameter with smaller assigned weight in generating symphonic longevity plan 124. Symphonic longevity plan disclosed here will be described in further detail below. As used in this disclosure, a “focus” is a center of interest of user such as, without limitation, a goal, a target, an intension, and the like thereof. In some cases, focus may include, without limitation, improving life energy measurement, improving health age, improving longevity age, improving performance age, and the like thereof. In some cases, focus may be determined manually by user, such as by a user input. In other cases, focus may be determined automatically by processor 104. In some embodiments, processor 104 may compare a plurality of compositional longevity parameters and determine focus as a function of the comparison. As a non-limiting example, processor 104 may determine a focus for improving a lowest compositional longevity parameter 112. As another non-limiting example, processor 104 may determine a focus by comparing compositional longevity parameters 112 to average compositional longevity parameters for similar (in age, weight, health, and/or the like) users. In this example, processor 104 may deem the longevity parameter 112 that is farthest from the average longevity parameter to be a focus. Additionally, or alternatively, the generation of the symphonic longevity plan 124 may further include assigning and/or updating weights (wᵢ) to different compositional longevity parameters (CLPᵢ) based on the user's interest, focus, goal, or intension: CLP_{weighted}=wᵢ×CLPᵢ. In an embodiment, the further the weight is away from 0, the greater or lesser effect will be on the CLPᵢ. A weight that is greater than 0 has a positive effect on the CLPᵢ. A weight that is lesser than 0 has a negative effect on the CLPᵢ. The user's interest may include, but is not limited to, enhancing longevity, equilibration of life energy, and boosting abilities. In a non-limited example, the user's goal is to feel joy, therefore a large positive weight will be assigned to the compositional longevity parameter 112 that include the life energy measurement during the generation of the symphonic longevity plan 124. The symphonic longevity plan 124 will then encompass increasing the life energy of the user. In an embodiment, symphonic longevity plan 124 may be stored in data storage device 128, such as compositional longevity database 300 disclosed with reference to FIG. 3.

With continued reference to FIG. 1, in some embodiments, generating symphonic longevity plan 124 may include generating symphonic longevity plan 124 in a bottom-up approach; for instance, without limitation, symphonic longevity plan 124 may be configured to treat illness and/or disease from bottom up. Symphonic longevity plan 124 may include a plurality of instructions or descriptions designed to treat illness and/or disease from the root, eliminate symptoms of illness and/or disease, and thereby reduce the probability of recurrence of such illness and/or disease. In some embodiments, processor 104 may generate the symphonic longevity plan 124 using a machine-learning process 116 trained with compositional longevity training data 120. Compositional longevity training data is a plurality of data entries containing a plurality of inputs that are correlated to a plurality of outputs for training a processor by a machine-learning process to determine the symphonic longevity plan 124 for the user. In an embodiment, the inputs of the compositional longevity training data 120 may contain one or more compositional longevity parameters 112 and the outputs of the compositional longevity training data 120 may contain symphonic longevity plan 124. In some cases, one or more compositional longevity parameters 112 may include, without limitation, life energy measurement, health age, longevity age, performance age, any combination of these, and the like thereof. In a non-limiting example, inputs of compositional longevity training data may include a plurality of life energy measurements and a plurality of performance age and outputs of compositional longevity training data 120 may include symphonic longevity plan 124. In some cases, compositional longevity training data may be obtained from data storage device 128 such as, without limitation, compositional longevity database 300. In some embodiments, compositional longevity training data 120 may include manually labeled data. As a non-limiting example, compositional longevity parameter 112, and/or symphonic longevity plan 124 may be manually collected and labeled by the user and/or a medical professional. As a non-limiting example, a medical professional may receive examples of compositional longevity parameter 112 and be asked to prescribe a symphonic longevity plan 124; this dataset may then be used as compositional longevity training data 120. In some embodiments, compositional longevity training data 120 may be derived from examples including a plurality of longevity measurement 108. As a non-limiting example, compositional longevity training data 120 may be chosen from real-life examples of longevity measurements 108 for patients and the associated compositional longevity parameter 112 of those patients.

With continued reference to FIG. 1, in some embodiments, processor 104 may be designed and configured to create a machine-learning model (i.e., machine-learning process 116) using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm mounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g., a quadratic, cubic or higher-order equation) providing a best predicted output/actual output fit is sought; similar methods to those described above may be applied to minimize error functions, as will be apparent to persons skilled in the art upon reviewing the entirety of this disclosure.

With continue reference to FIG. 1, the symphonic longevity plan 124 may identify a set of actions designed to balance, or/and improve the compositional longevity parameter 112 of the user. In some cases, set of actions may include guidance, interventions, or/and rectification including, but is not limited to, diet correction, physical fitness, supplementation, health testing to identify root cause of a problem, and the like. In a non-limited example, the symphonic longevity plan 124 may suggest adding adequate protein in user's diet to increase the health age of the user to an optimal metric. In an embodiment, the one or more actions identified by the symphonic longevity plan 124 may be designed to correct one or more deficiencies of the user. As used in this disclosure, a “deficiency” is a physical or a mental area, aspect, or field that lack one or more substance or/and being deficient or incomplete. In a non-limited example, the symphonic longevity plan 124 may provide detailed instructions of moderate aerobic exercise for the user with insomnia to relieving the symptom and improve user's sleep. Symphonic longevity plan 124 may be updated at any given time interval such as, without limitation, hourly, daily, weekly, bi-weekly, monthly, and the like thereof. Process of updating symphonic longevity plan 124 and/or any process described in this disclosure may be performed, without limitation, at any given time interval.

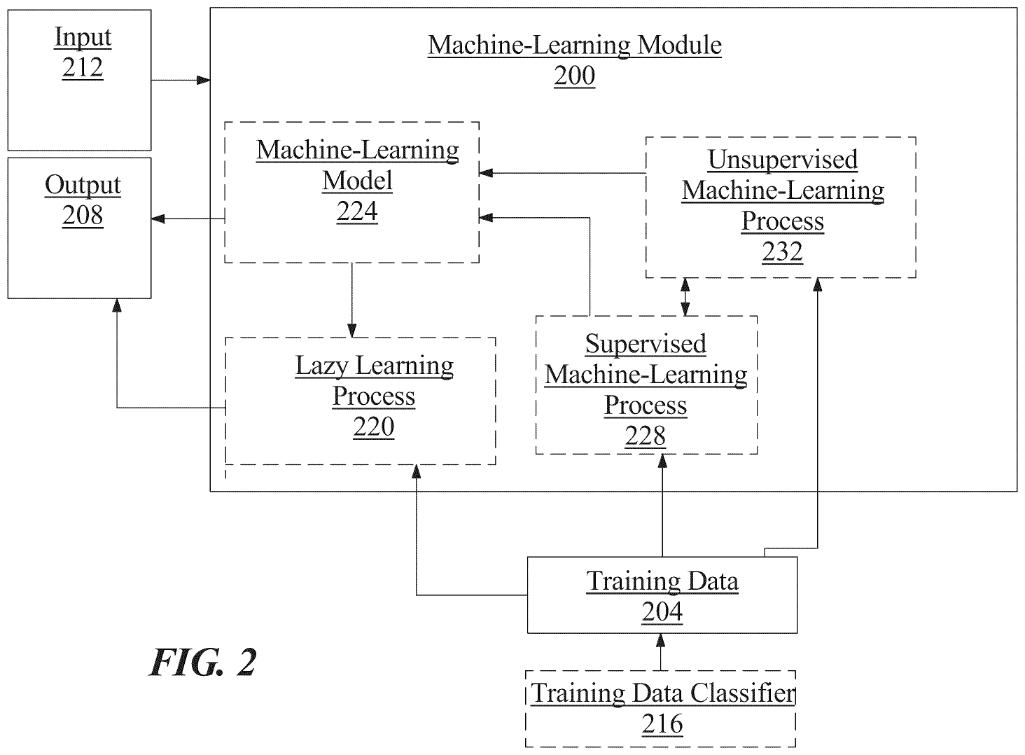

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example inputs may include longevity measurement 108 and outputs may include compositional longevity parameter 112. As another non-limiting example, inputs may include compositional longevity parameter 112 and/or longevity measurement 108 and outputs may include symphonic longevity plan 124.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 200 may generate a classifier using a classification algorithm, defined as a process whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to life energy measurement, health age, longevity age, performance age, compositional longevity parameter, and symphonic longevity plan 124.

With continued reference to FIG. 1, A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Processor 104 and/or another device may generate a classifier using a classification algorithm, defined as a processes whereby a processor 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

With continued reference to FIG. 1, processor 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Processor 104 may then calculate a likelihood table by calculating probabilities of different data entries and classification labels. Processor 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, processor 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=√Σᵢ₌₀ⁿaᵢ², where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively, or additionally, and with continued reference to FIG. 2, machine-learning processes as described in this disclosure may be used to generate machine-learning models 224. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs as described above in this disclosure as inputs, outputs as described above in this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Referring now to FIG. 3, a non-limiting exemplary embodiment of a compositional longevity database 300 is illustrated. Processor 104 may be communicatively connected with compositional longevity database 300. For example, in some cases, compositional longevity database 300 may be local to processor 104. Alternatively, or additionally, in some cases, compositional longevity database 300 may be remote to processor 104 and communicative with processor 104 by way of one or more networks. Network may include, but not limited to, a cloud network, a mesh network, or the like. By way of example, a “cloud-based” system, as that term is used herein, can refer to a system which includes software and/or data which is stored, managed, and/or processed on a network of remote servers hosted in the “cloud,” e.g., via the Internet, rather than on local severs or personal computers. A “mesh network” as used in this disclosure is a local network topology in which the infrastructure processor 104 connect directly, dynamically, and non-hierarchically to as many other computing devices as possible. A “network topology” as used in this disclosure is an arrangement of elements of a communication network. Database may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

Referring now to FIG. 3, at least the processor 104 may, alternatively or additionally, store and/or retrieve data from a longevity measurement table 304, compositional longevity parameter table 308, and symphonic longevity plan table 312. Determinations by a machine learning process may also be stored and/or retrieved from the compositional longevity database 300, for instance in non-limiting examples a misreporting factor. As a non-limiting example, compositional longevity database 300 may organize data according to one or more compositional longevity database 300 tables. One or more database tables may be linked to one another by, for instance in a non-limiting example, common column values. For instance, a common column between two tables of database may include an identifier of a submission, such as a form entry, textual submission, research paper, or the like, for instance as defined below; as a result, a query may be able to retrieve all rows from any table pertaining to a given submission or set thereof. Other columns may include any other category usable for organization or subdivision of expert data, including types of expert data, names and/or identifiers of experts submitting the data, times of submission, or the like; persons skilled in the art, upon reviewing the entirety of this disclosure, will be aware of various ways in which data from one or more tables may be linked and/or related to data in one or more other tables.

Still referring to FIG. 3, in a non-limiting embodiment, one or more compositional longevity database tables of a database may include, as a non-limiting example, a longevity measurement table 304, which may include longevity measurement for use in identifying longevity measurement of a user and/or correlating longevity marker data, entries indicating values and/or degrees of relevance to and/or efficacy in identifying longevity measurement pertaining to a user, and/or other elements of data processor 104 and/or apparatus 100 may use to determine values and/or usefulness and/or relevance of longevity marker data in identifying longevity measurements as described in this disclosure. One or more tables may include a longevity measurement table 304, which may correlate longevity markers and/or combinations thereof to one or more longevity measurements; longevity measurement table 304 may contain a plurality of entries associating at least an element of longevity marker with longevity measurement. One or more tables may include, without limitation, a compositional longevity parameter table 308, which may contain one or more inputs identifying one or more categories of data, for instance demographic data, medical history data, physiological data, or the like. One or more tables may include a compositional longevity parameter table 308, which may contain one or more entries indicating compositional longevity parameters pertaining to the user. In some embodiments, without limitation, compositional longevity parameter table 308 may include one or more columns contain life energy measurement pertain to users. In some embodiments, without limitation, compositional longevity parameter table 308 may include one or more columns contain health age pertain to users. In some embodiments, without limitation, compositional longevity parameter table 308 may include one or more columns contain longevity age pertain to users. In other embodiments, without limitation, compositional longevity parameter table 308 may include one or more columns contain performance age pertain to users. Additionally, or alternatively, one or more tables may include, without limitation, a symphonic longevity plan table 312, which may include symphonic longevity plan for use in correlating one or more users. One or more tables may include a symphonic longevity plan table 312, which may correlate compositional longevity parameters and/or combinations thereof to one or more longevity measurements.

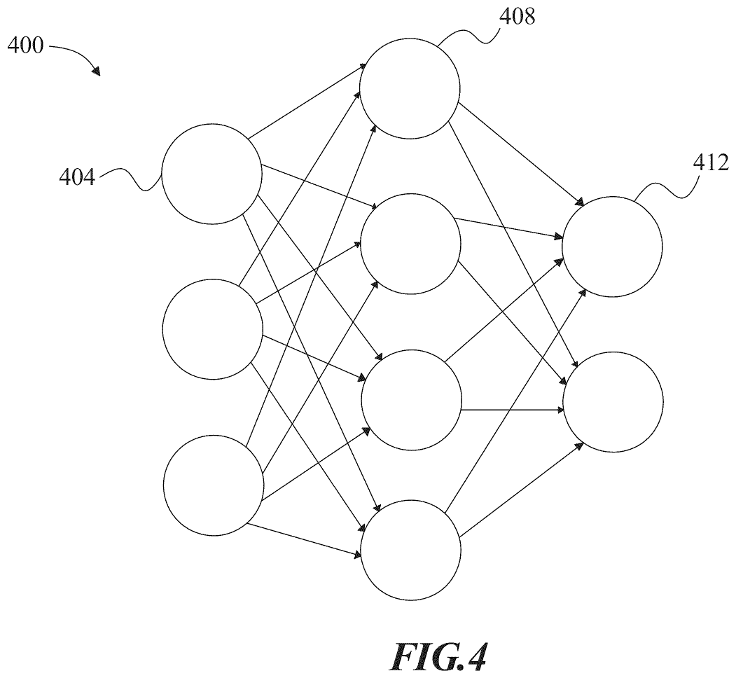

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

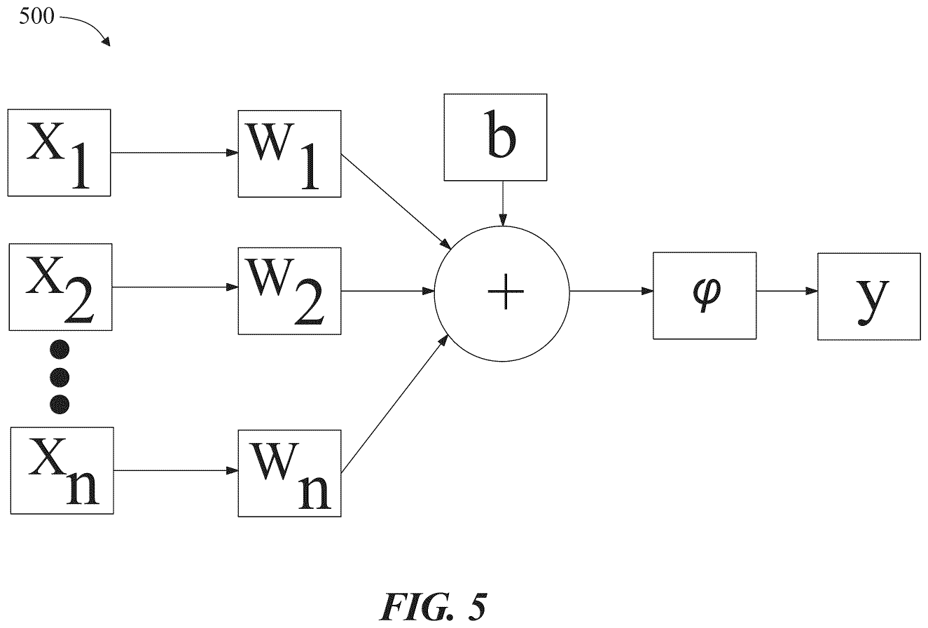

Referring now to FIG. 5, an exemplary embodiment of a node of a neural network is illustrated. A node may include, without limitation a plurality of inputs xᵢ that may receive numerical values from inputs to a neural network containing the node and/or from other nodes. Node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ, applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

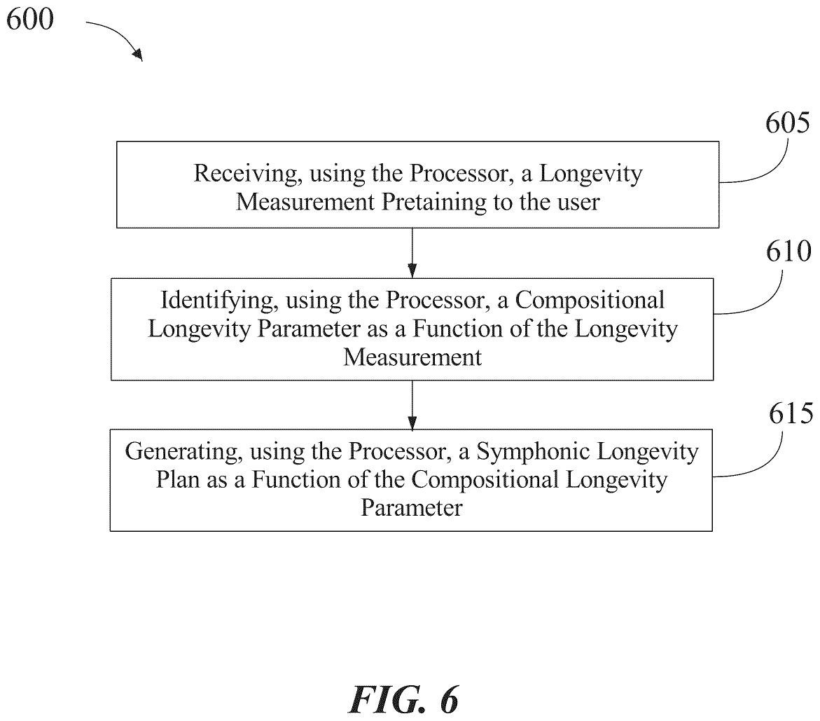

Referring now to FIG. 6 an exemplary method 600 for enhancing longevity is illustrated. Method 600 includes a step 605, of receiving, using a processor, a longevity measurement pertaining to a user, without limitation, as described above in reference to FIGS. 1-5. In some embodiments, the longevity measurement may include a longevity marker associated with the user. Longevity marker associated with user are described further with respect to FIG. 1.

With continued reference to FIG. 6, method 600 includes a step 610 of identifying, using the processor, a compositional longevity parameter as a function of the longevity measurement. This may be implemented, without limitation, as described above with reference to FIGS. 1-5. In some embodiments, the compositional longevity parameter may include a life energy measurement related to the user. In some embodiments, the compositional longevity parameter may include a health age related to the user. In some embodiments, the compositional longevity parameter may include a longevity age related to the user. In some embodiments, the compositional longevity parameter may include a performance age related to the user. This may be implemented, without limitation, as described above with reference to FIGS. 1-5. In some embodiments, compositional longevity parameter may further include an assigned weight. In some embodiments, the identification the compositional longevity parameter may include training and using a machine-learning process using longevity training data. This may be implemented, without limitation, as described above with reference to FIGS. 1-5.

With continued reference to FIG. 6. Method 600 includes a step 615 of generating, using the processor, a symphonic longevity plan as a function of the compositional longevity parameter. This may be implemented, without limitation, as described above with reference to FIGS. 1-5. In some embodiments, the symphonic longevity plan may identify one or more problematic areas. In some embodiments, the generation of the symphonic longevity plan may include weighting different compositional longevity parameters. In some embodiments, the generation of the symphonic longevity plan may also include training and using a machine-learning process using compositional longevity training data. This may be implemented, without limitation, as described above with reference to FIGS. 1-5.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

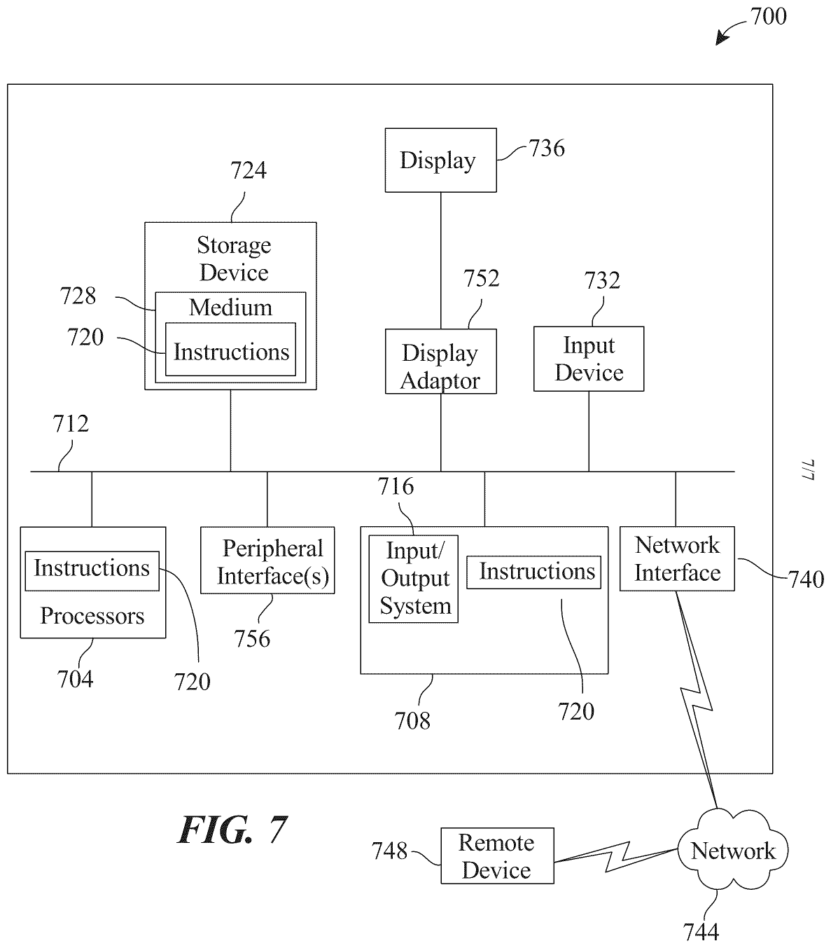

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for enhancing longevity, wherein the apparatus comprises:
at least a processor; and
a memory communicatively connected to the at least a processor, the memory containing instructions configuring the at least a processor to:
receive a longevity measurement pertaining to a user, wherein the longevity measurement comprises one or more longevity markers, and wherein the one or more longevity markers comprises telomerase length;
identify a compositional longevity parameter, as a function of the longevity measurement;
generate a symphonic longevity plan pertaining to the user as a function of the compositional longevity parameter, wherein generating the symphonic longevity plan further comprises:
training a machine-learning process using compositional longevity training data, wherein training the machine-learning process further comprises:
generating a training data classifier configured to classify training data to a compositional longevity parameter classification utilizing a classification algorithm, wherein the classification algorithm comprises a supervised machine-learning process that iteratively derives the training data classifier, and wherein the compositional longevity parameter classification comprises data indicating an age of systems associated with the user's longevity, and wherein the compositional longevity training data contains a plurality of inputs containing compositional longevity parameters correlated to a plurality of outputs containing symphonic longevity plans; and
training the machine-learning process using the classified compositional longevity training data;
generating the symphonic longevity plan pertaining to the user as a function of the trained machine-learning process; and
update the symphonic longevity plan after a time interval based on a deficiency in the compositional longevity parameter, wherein the compositional longevity parameter comprises a life energy measurement which includes a mental health status of the user regarding a relationship health.

2. (canceled)

3. The apparatus of claim 1, wherein the compositional longevity parameter further comprises a health age.

4. The apparatus of claim 1, wherein the compositional longevity parameter further comprises a longevity age.

5. The apparatus of claim 1, wherein the compositional longevity parameter further comprises a performance age.

6. The apparatus of claim 1, wherein the compositional longevity parameter further comprises an assigned weight.

7. The apparatus of claim 6, wherein generating the symphonic longevity plan further comprises:
assigning a first compositional longevity parameter a first weight;
assigning a second compositional longevity parameter a second weight; and
generating the symphonic longevity plan as a function of the first weight and the second weight.

8. The apparatus of claim 1, wherein the symphonic longevity plan identifies a problematic area of the user.

9. The apparatus of claim 1, wherein the symphonic longevity plan identifies a set of actions designed to improve the compositional longevity parameter and correct the deficiency of the user.

10. The apparatus of claim 1, wherein identifying the compositional longevity parameter pertaining to the user further comprises:
training an additional machine-learning process using longevity training data, wherein the longevity training data contains a plurality of inputs containing longevity measurements correlated to a plurality of outputs containing compositional longevity parameters; and
generating the compositional longevity parameter pertaining to the user as a function of the trained additional machine-learning process.

11. A method for enhancing longevity, wherein the method comprises:
receiving, using a processor, a longevity measurement pertaining to a user, wherein the longevity measurement comprises one or more longevity markers, and wherein the one or more longevity markers comprises telomerase length;
identifying, using the processor, a compositional longevity parameter, as a function of the longevity measurement;
generating, using the processor, a symphonic longevity plan pertaining to the user as a function of the compositional longevity parameter, wherein the generating the symphonic longevity plan further comprises:
training a machine-learning process using compositional longevity training data, wherein training the machine-learning process further comprises:
generating a training data classifier configured to classify training data to a compositional longevity parameter classification utilizing a classification algorithm, wherein the classification algorithm comprises a supervised machine-learning process that iteratively derives the training data classifier, and wherein the compositional longevity parameter classification comprises data indicating an age of systems associated with the user's longevity, and wherein the compositional longevity training data contains a plurality of inputs containing compositional longevity parameters correlated to a plurality of outputs containing symphonic longevity plans; and
training the machine-learning process using classified compositional longevity training data;
generating the symphonic longevity plan pertaining to the user as a function of the trained machine-learning process; and
updating the symphonic longevity plan after a time interval based on a deficiency in the compositional longevity parameter, wherein the compositional longevity parameter comprises a life energy measurement which includes a mental health status of the user regarding a relationship health.

12. (canceled)

13. The method of claim 11, wherein the compositional longevity parameter further comprises a health age.

14. The method of claim 11, wherein the compositional longevity parameter further comprises a longevity age.

15. The method of claim 11, wherein the compositional longevity parameter further comprises a performance age.

16. The method of claim 11, wherein the compositional longevity parameter further comprises an assigned weight.

17. The method of claim 16, wherein generating the symphonic longevity plan further comprises:
assigning a first compositional longevity parameter a first weight;
assigning a second compositional longevity parameter a second weight; and
generating the symphonic longevity plan as a function of the first weight and the second weight.

18. The method of claim 11, wherein the symphonic longevity plan identifies a problematic area of the user.

19. The method of claim 11, wherein the symphonic longevity plan identifies a set of actions designed to improve the compositional longevity parameter and correct the deficiency of the user.

20. The method of claim 11, wherein identifying the compositional longevity parameter pertaining to the user further comprises:
training an additional machine-learning process using longevity training data, wherein the longevity training data contains a plurality of inputs containing longevity measurements correlated to a plurality of outputs containing compositional longevity parameters; and
generating the compositional longevity parameter pertaining to the user as a function of the trained additional machine-learning process.

21. The apparatus of claim 1, wherein the life energy measurement further includes a life energy comparison metric.

22. The method of claim 11, wherein the life energy measurement further includes a life energy comparison metric.
